(19) United States
(12) Patent Application Publication
(10) Pub. No.: US 2023/0200966 A1
(43) Pub. Date: Jun. 29, 2023

(54) TISSUE WRAP DEVICE WITH ATTACHMENT FEATURES

(71) Applicant: Axogen Corporation, Alachua, FL (US)

(72) Inventors: Brian ROMOT, Alachua, FL (US); Angelo SCOPELIANOS, Alachua, FL (US); Robert C. DILUCCIO, Alachua, FL (US)

(73) Assignee: Axogen Corporation, Alachua, FL (US)

(21) Appl. No.: 18/051,353

(22) Filed: Oct. 31, 2022

Related U.S. Application Data
(60) Provisional application No. 63/294,156, filed on Dec. 28, 2021.

(51) Int. Cl. A61F 2/00; A61M 37/00; A61B 17/00; A61L 27/26; A61L 27/36; B33Y 80/00; B29C 65/48; B29C 65/00

(52) U.S. Cl. CPC A61F 2/0077; A61B 17/00491; A61L 27/26; A61L 27/3604; A61L 27/3629; A61M 37/0015; B29C 65/48; B29C 66/47; B33Y 80/00; A61F 2002/0081; A61F 2220/0016; A61F 2240/001; A61M 2037/0023; B29K 2071/00

(57) ABSTRACT

The present disclosure provides tissue wrap devices with embedded three dimensional attachment features, methods of manufacturing three-dimensional attachment features for tissue wrap devices, and methods of using a nerve wrap device to protect a damaged nerve. Tissue wrap devices of the present disclosure may include a sheet of biocompatible material, the sheet having a first side, a second side, a middle portion, a first outer portion, and a second outer portion, the first side of the first outer portion being configured to overlap and interface with the second side of the second outer portion when the sheet is transitioned to a rolled configuration; wherein the first side of the first outer portion comprises a plurality of three dimensional attachment features; and wherein the plurality of three dimensional attachment features are configured to engage with the second side of the second outer portion to maintain the sheet in the rolled configuration.

### CROSS-REFERENCE TO RELATED APPLICATION

This patent application claims the benefit of priority of U.S. Provisional Pat. Application No. 63/294,156, filed on Dec. 28, 2021, which is incorporated by reference herein in its entirety.

### TECHNICAL FIELD

Various embodiments of the present disclosure relate generally to tissue wraps and, more particularly, to nerve wraps with attachment features incorporated therein.

### BACKGROUND

Following one or more of tissue injury, tissue repair, and/or tissue reconstruction, protecting a damaged tissue area may facilitate the healing process. For example, in the case of nerve tissue, failure to cover and/or isolate a nerve repair or nerve injury site may lead to undesired axonal growth into surrounding areas, which may result in soft tissue attachment and scarring. By protecting a nerve repair or injury site (e.g., through covering and isolation), undesired axonal growth may be inhibited (e.g., reduced or eliminated), and, in some instances, decreased healing time may be achieved by directing axonal growth towards a preferred nerve regeneration site, instead of non-targeted areas. Further, such techniques can also provide reinforcement to a nerve repair or injury site and inhibit separation of coapted nerves. In order to provide protection and covering at a nerve repair or injury site, membranous tissue grafts in the form of tubes, conduits, sheets for wrapping (i.e., wraps), or other forms for supporting and reinforcing microsurgical repairs of injured nerves may be used.

Conventional membranous tissue grafts used to cover and protect injured and/or compressed nerves are typically formed in accordance with specific nerve diameters. Thus, grafts in the form of wraps or sheets may be selected based on the diameter of the injured nerve. However, such configurations may be limited to specific nerve sizes.

Even when a membranous tissue graft has been configured to cover and surround the injured nerves (e.g., wrap around the injured nerves), the failure to properly secure, anchor, and/or hold the graft together may result in the migration of the graft from the area intended to be protected, as well as the exposure of said area. A common technique for securing a membranous tissue graft, such as a tissue graft in the form of a wrap, is suturing. However, suturing involves the use of materials (e.g., needles) that may tear or rip the tissue or tissue graft during the securing process, and, in the case of nerve tissue, may damage the epineurium of the nerve leading to further damage. As a result, suturing as a means to secure and hold a membranous tissue graft in the form of a wrap together may be time intensive and in some instances, may lead to further damage.

There is a need, therefore, for membranous tissue grafts, such as tissue grafts in the form of nerve wraps, that have attachment features that allow the graft to adhere to itself and/or the epineurium, provide a customized fit around nerve tissue, and eliminate or at reduce the need for sutures.

The present invention is directed to overcoming one or more of these above-referenced challenges.

### SUMMARY OF THE DISCLOSURE

According to certain aspects of the disclosure, a tissue wrap (e.g., nerve wrap) device may include attachment features incorporated therein. In particular, the attachment features may be three dimensional attachment features.

In one aspect, a tissue wrap device may include a sheet of biocompatible material. The sheet may have a first side, a second side, a middle portion, a first outer portion, and a second outer portion. The first side of the first outer portion may be configured to overlap and interface with the second side of the second outer portion when the sheet is transitioned to a rolled configuration. The first side of the first outer portion may include a plurality of three dimensional attachment features and the plurality of three dimensional attachment features may be configured to engage with the second side of the second outer portion to maintain the sheet in the rolled configuration.

A method of repairing a tissue may include wrapping the tissue wrap device around the tissue and overlapping the first side of the first outer portion with the second side of the second outer portion, wherein the plurality of three dimensional attachment features adheres the first side of the first outer portion to the second side of the second outer portion to maintain the tissue wrap device in the rolled configuration.

In another aspect, a method of manufacturing a plurality of three-dimensional attachment features for a tissue wrap device may include three dimensional (3D) printing a biocompatible material to form a plurality of three dimensional attachment features. The biocompatible material may include one or more of poly(ethylene glycol) diacrylate, polyurethane, polyurethane/urea, poly(glycolic acid), poly(lactic acid), poly(lactic-co-glycolic acid), polycaprolactone, agarose, alginate, chitosan, collagen, fibrin, gelatin, hyaluronic acid, gelatin methacryloyl, polyethylene glycol, or a mixture thereof. The three dimensional attachment features may have an average diameter of about 25 µm to about 75 µm and a height of about 1000 µm or less.

In another aspect, a method of using a nerve wrap device to protect a damaged nerve may include positioning the nerve wrap device relative to the damaged nerve; wrapping the nerve wrap device around the nerve so that a first portion of the nerve wrap device overlaps a second portion of the nerve wrap device, and forming a rolled configuration of the nerve wrap device. At least one of the first portion and the second portion may include a plurality of three dimensional attachment features, wherein the plurality of three dimensional attachment features secures the first portion and the second portion to each other to maintain the rolled configuration.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments. Drawings included herein may not be drawn to scale.

FIG. 1A is a schematic view of a tissue wrap in the form of a sheet.

FIG. 1B is a schematic view of a tissue wrap in a rolled configuration.

FIG. 2 is a schematic view of a tissue wrap, having attachment features, for use with an injured nerve, according to one or more embodiments.

FIGS. 3A-3D and FIGS. 4-6 show various configurations of nerve wraps having attachment features, according to one or more embodiments, with FIG. 3A showing a nerve wrap with hook and loop interlocking features as well as microneedles for contact with the nerve, with FIG. 3B showing a nerve wrap with hook and loop interlocking features, with FIG. 3C showing a nerve wrap with hook and loop interlocking features as well as microneedles for contact with the nerve, with FIG. 3D showing a nerve wrap with hook and loop interlocking features as well as microneedles for contact with the nerve, with FIG. 4 showing a nerve wrap with ball and socket interlocking features as well as microneedles for contact with the nerve, with FIG. 5 showing a nerve wrap with barbed microneedle attachment features as well as microneedles for contact with the nerve, and with FIG. 6 showing a nerve wrap with hook and loop interlocking features as well as barbed microneedles for contact with the nerve.

FIG. 7, illustrates, in flow chart form, an exemplary method of using the nerve wrap device, according to one or more embodiments.

### DETAILED DESCRIPTION OF EMBODIMENTS

Various embodiments of the present disclosure relate generally to tissue wraps and, more particularly, to tissue wraps with attachment features.

The singular forms “a,” “an,” and “the” include plural reference unless the context dictates otherwise. The terms “approximately” and “about” refer to being nearly the same as a referenced number or value. As used herein, the terms “approximately” and “about” generally should be understood to encompass ± 10% of a specified amount or value. As used herein, the terms “comprises,” “comprising,” “including,” “having,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” is used herein in the sense of “example,” rather than “ideal.” In addition, the term “between” used in describing ranges of values is intended to include the minimum and maximum values described herein. The use of the term “or” in the claims and specification is used to mean “and/or” unless explicitly indicated to refer to alternatives only or the alternatives are mutually exclusive, although the disclosure supports a definition that refers to only alternatives and “and/or.”

Although embodiments of the disclosure are described in relation to wrap devices for reconstruction or repair of a peripheral nerve injury, it is contemplated that embodiments of the disclosure may be used with other suitable types of tissue. For example, embodiments of the disclosure may be used with and/or applied to, e.g., epithelial tissue, connective tissue, vascular tissue, dermal tissue, skeletal tissue, muscle tissue, cardiac tissue, lung tissue, urological tissue, ligament tissue, adipose tissue, connective tissue, or nerve tissue. Accordingly, the terms “nerve” and “nerve tissue” as used herein are used to describe any tissue to which the embodiments of the present disclosure may be applied. Thus, as used herein, the term “nerve wrap” and “nerve wrap device” describe a wrap device suitable for use with any tissue.

The term “membranous tissue graft” as used herein may generally refer to a biocompatible graft suitable for implantation into a subject in a surgical procedure or other medical procedure. A membranous tissue graft may include synthetic or biological tissue, and, if biological tissue, may include human or animal tissue. In some examples, suitable grafts may be formed of human or animal, e.g., porcine, small intestine submucosa (SIS). Examples of suitable membranous tissue grafts include the Avive® Soft Tissue Membrane from Axogen, Inc (Alachua, FL, US) and the Axoguard Nerve Protector®.

The term “embedded” as used herein may generally refer to one object being fixed at, on, or beneath the surface of another object.

The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed.

The tissue wrap devices of the present disclosure may be prepared from membranous tissue grafts and may be formed with attachment features embedded therein. The attachment features may allow the tissue wrap device to adhere to itself and/or the tissue to which it is applied. For example, in the case of nerves, the attachment features may allow a nerve wrap device to adhere to itself and/or the epineurium, when wrapped around nerve tissue. The present disclosure, therefore, may facilitate the protection of nerve tissue after surgery, by use of a nerve wrap device that may stay in place at the nerve site and may be held together via embedded attachment features for a period of time before degrading, and may reduce or eliminate the need for suturing. Exemplary nerve wrap devices with attachment features, related methods for their preparation, and related methods of their use are described in detail below.

According to some embodiments of the present disclosure, tissue wraps, e.g., nerve wraps, may be prepared from membranous tissue grafts. The membranous tissue grafts may be formed in a sheet, and may be made up of one or more layers of SIS.

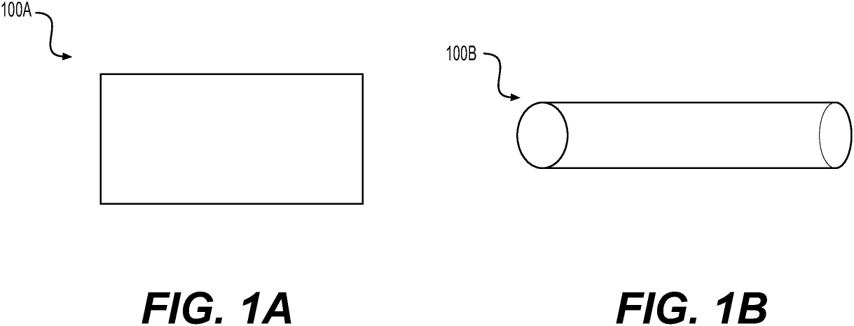

FIGS. 1A-1B depict an example nerve wrap 100 prior to the inclusion of attachment features. Nerve wrap 100 of the present disclosure may be a membranous tissue graft. The membranous tissue graft used for nerve wrap 100 may have a thickness of, for example, from about 25 microns to about 3 mm, about 100 microns to about 2.75 mm, about 200 microns to about 2.5 mm, about 300 microns to about 2 mm, or about 500 microns to about 1.5 mm. Further, the membranous tissue graft used for nerve wrap 100 may be prepared from a synthetic material, a natural material, or combinations thereof. Nerve wrap 100—or a nerve wrap as described in any of the embodiments herein-may be in the form of a substantially rectangular sheet (as shown in FIG. 1A), a circular sheet, or a sheet having other regular or irregular shapes. While one sheet is shown in FIG. 1A, nerve wrap 100 may include a plurality of sheets stacked and secured together.

Suitable synthetic materials include, but are not limited to, one or more of silicone membranes, expanded polytetrafluoroethylene (ePTFE), polyethylene tetraphthlate (Dacron), polyurethane aliphatic polyesters, poly(amino acids), poly(propylene fumarate), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, and blends thereof.

Suitable natural materials include, but are not limited to, one or more of collagen, elastin, thrombin, fibronectin, starches, poly(amino acid), gelatin, alginate, pectin, fibrin, oxidized cellulose, chitin, chitosan, tropoelastin, hyaluronic acid, fibrin-based materials, collagen-based materials, hyaluronic acid-based materials, glycoprotein-based materials, cellulose-based materials, silks and combinations thereof. Suitable natural materials, may also include cellularized or decellularized tissue constructs (e.g., demineralized bone, submucosa extracellular matrix (ECM), small intestine submucosa (SIS), dermis, muscle, fascia, or birth tissue, such as amnion).

In some embodiments, a nerve wrap of the present disclosure (e.g., nerve wrap 100), may be made from small intestine submucosa (SIS) material, such as porcine small intestine submucosa. In particular, FIG. 1A shows a sheet configuration 100A of nerve wrap 100. Nerve wrap 100 may be in the form of a rectangle, having a length that is longer than a width, measured in a direction perpendicular to the length. A length of nerve wrap 100 may be within a range of about 5 mm to about 60 mm, within a range of about 10 mm to about 50 mm, or within a range of about 20 mm to about 40 mm, for example. A width of nerve wrap 100 may be within a range of about 1 mm to about 20 mm, within a range of about 5 mm to about 15 mm, within a range of about 5 mm to about 10 mm, e.g., about 2 mm, about 5 mm, about 7 mm, or about 10 mm. Exemplary dimensions of the sheet configuration 100A of nerve wrap 100 may have a length of about 40 mm and a width of about 10 mm, a length of about 40 mm and a width of about 7 mm, or a length of about 40 mm and a width of about 5 mm.

Nerve wrap 100, having a sheet configuration 100A may be wrapped into a rolled configuration 100B, as shown in FIG. 1B. In other embodiments, a nerve wrap may come pre-rolled in the rolled configuration 100B, e.g., and may come fully formed as a tube or cylinder. The nerve wrap 100 in the form of a sheet configuration 100A may be wrapped around an injured or damaged nerve to produce a rolled configuration 100B of the nerve wrap. The rolled configuration 100B may provide nerve wrap 100 with a tubular or cylindrical shape. The tubular or cylindrical shape of the rolled configuration 100B may define a diameter within a range of about 0.5 mm to about 10 mm, about 1 mm to about 8 mm, or about 1.5 mm to about 7 mm. In particular, a diameter may be equal to about 1.5 mm, about 2 mm, about 3 mm, about 3.5 mm, about 4 mm, about 5 mm, about 6 mm, about 7 mm, or about 10 mm. Wrapping a nerve wrap of the present disclosure around an injured or damaged nerve may produce a tube having a size (diameter, length) of about 2 mm x about 20 mm, about 3.5 mm x about 20 mm, about 5 mm x about 20 mm, about 7 mm x about 20 mm, about 10 mm x about 20 mm, about 3.5 mm x about 40 mm, about 5 mm x about 40 mm, about 7 mm x about 40 mm, or about 10 mm x about 40 mm. All of the above dimensions are exemplary, and the dimensions of the present disclosure are not limited thereto. For example, when the nerve wrap 100 in sheet configuration 100A is wrapped around a nerve, the rolled configuration 100B of the nerve wrap 100 may have a length corresponding with the length of sheet configuration 100A, while the diameter may vary depending on the extent to which the sides of the rectangle are overlapped with each other when forming the tubular configuration of 100B. The diameter of nerve wrap 100 in the rolled configuration 100B may surround the nerve around which it is wrapped. Thus, the diameter of nerve wrap 100 in rolled configuration 100B may correspond approximately to the diameter of the nerve that nerve wrap 100 surrounds.

The nerve wrap 100 as depicted in FIG. 1A is an unmodified nerve wrap. An unmodified nerve wrap prepared from membranous tissue graft, when displayed in sheet configuration 100A may have a relatively smooth or flat, top and bottom surface. The term “unmodified nerve wrap” as used herein may refer to nerve wraps that do not include one or more attachment features. However, unmodified nerve wraps, such as nerve wrap 100 shown in sheet configuration 100A, may be modified to include one or more attachment features according to embodiments of the present disclosure. When embedded onto or within the surface(s) of nerve wraps of the present disclosure, the attachment features may enable the nerve wrap, after being wrapped around nerve tissue, to adhere to itself and/or the epineurium, and to remain in a rolled configuration around the nerve tissue such as the rolled configuration 100B.

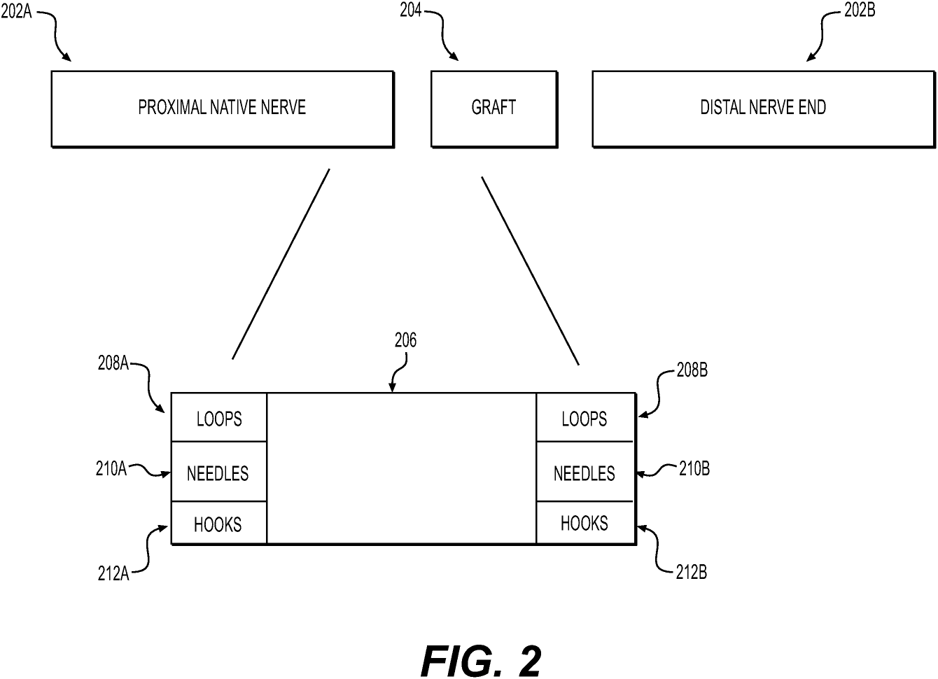

FIG. 2 shows a schematic diagram according to the present disclosure of how the nerve wrap device 206 with attachment features may interact with an injured nerve. Compared to an unmodified nerve wrap (i.e., without attachment features), such as nerve wrap 100 as depicted in FIG. 1A, a nerve wrap device 206 with attachment features may have a similar length but may be relatively wider to allow for the addition of the attachment features on portions of the wrap that will overlap with each other in order to fasten the two sides together in the rolled configuration. Nerve wrap devices according to the present disclosure may be, for example, about 0.25 mm to about 5 mm wider, about 0.5 mm to about 4 mm wider, about 1 mm to about 3 mm wider, or about 2 mm wider, for example along portions on one or more sides of the nerve wrap. In other words, the nerve graft device 206 includes excess tissue graft material on one or more sides that extends beyond the portions of the wrap that are configured to contact the injured nerve tissue. The excess tissue graft material on one or more sides that are configured to overlap are where the attachment features are located. The diameter of the nerve wrap devices 206 of the present disclosure may increase by about 1 mm or less, if at all, as compared to unmodified nerve wrap devices when in a tubular configuration.

For example, the middle portion of nerve wrap device 206 that aligns with needles 210A and needles 210B may extend outward by approximately an extra 1 mm on each side of the nerve wrap device 206. As shown in FIG. 2, the excess material used for nerve wrap device 206 allows for the inclusion of interlocking, attachment features along the outer portions of the wrap. As used herein, the terms “outer portion” or “outer portions” of nerve wrap devices refer to portions of the nerve wrap devices that are configured to overlap with one another in a rolled configuration. As used herein, the terms “middle portion” or “middle portions” used to refer to portions that are not configured to overlap with one another in a rolled configuration, and thus broadly refer to regions of the wrap devices located between the outer portions. “Middle portion(s)” may refer to portions of the nerve wrap devices that are configured to interface with the tissue when the nerve wrap devices are in a rolled configuration. Unmodified nerve wraps may be configured so that the when the wrap in sheet form is wrapped around the nerve, the outer, opposite side portions of the wrap are brought together to meet or align along with little, if any, overlap. Such configurations typically require the use of suture materials to form the wrapped, tube configuration, and to hold the wrap in place on the nerve tissue. However, the nerve wrap devices of the present disclosure, such as nerve wrap device 206, may be relatively wider to allow for the inclusion of attachment features and overlap of the opposite side portions to secure the tubular structure in place.

The ability to overlap provided by embodiments of the present disclosure, due to the use of excess material, enables the interlocking attachment features embedded along at least one of the overlapping portions of the nerve device to interface when the overlapping portions are brought together. Once interfaced, the overlapping portions and the embedded attachment features may attach, allowing the nerve wrap device to adhere to itself. In order to provide for the adherence mechanism described above, nerve wrap devices according to embodiments of the present disclosure may be prepared from membranous tissue grafts in sheet form that have a width that allows for sufficient overlap for wrapping and the engagement of the attachment features.

Nerve wrap device 206 may have corresponding attachment features along the outer portions that are configured to overlap and adhere to each other upon wrapping around the nerve. Exemplary attachment features that may be embedded on the overlapping portions according to certain embodiments of the present disclosure include hooks and loops as shown in FIG. 2. Attachment features may also be embedded on the middle or central portion of the nerve wrap that is located, e.g., between the overlapping portions. The middle portion attachment features may be included to attach the wrap to an outer portion of the tissue to which the wrap is applied to inhibit migration of the wrap and tissue relative to one another so that the wrap stays in place covering the injured portion of tissue. In the case of nerves, the tissue attachment features may attach and adhere to an outer portion of the nerve epineurium. Exemplary tissue attachment features include microneedles. For example, FIG. 2 shows that microneedles may be located on the sheet for nerve wrap device 206 between the loop and hook attachment features.

According to nerve wrap configuration 200, nerve wrap device 206 may be rolled onto itself to wrap around and surround an injured nerve having a proximal native nerve 202A and a distal nerve end 202B. When nerve wrap device 206 is rolled onto itself, a lumen may be created therein, and the injured nerve may be received within the lumen of the nerve wrap device 206. For example, when the proximal native nerve 202A and distal nerve end 202B are reconnected with a nerve graft 204 in between, nerve wrap device 206 in sheet form may be positioned and wrapped around the repaired nerve where proximal native nerve 202A, graft 204, and distal nerve end 202B meet. Nerve wrap device 206 may be positioned so that the middle portion of the wrap is placed around the nerve forming a covering, and the outer portions are wrapped around in an overlapping configuration. Therefore, microneedles 210A and 210B embedded on the central, end portions of nerve wrap device 206 may attach to a portion of the proximal native nerve 202A and a portion of distal nerve end 202B, respectively. Needles 210A and 210B may engage an outer portion of the epineurium of proximal native nerve 202A and distal nerve end 202B, helping to secure proximal native nerve 202A and distal nerve end 202B within nerve wrap device 206.

When the overlapping portions comprising loops 208A oriented nearest proximal native nerve 202A and loops 208B oriented nearest distal nerve end 202B are brought together with the overlapping portions comprising hooks 212A nearest proximal native nerve 202A and hooks 212B nearest distal nerve end 202B, the overlapping portions may engage and adhere to each other, enclosing the dimeter of the nerve. In at least one embodiment, in which a gap exists between proximal native nerve 202A and distal nerve end 202B, a graft such as graft 204, may be used to connect proximal native nerve 202A and distal nerve end 202B. Nerve wrap device 206 may then be wrapped around the nerve connected by graft 204 in accordance with the configuration described above. Although FIG. 2 depicts the use of graft 204 used between transected proximal native nerve 202A and distal nerve end 202B, nerve wrap device 206 may be used to wrap around only proximal native nerve 202A and distal nerve end 202B, without the use of nerve graft 204, or may be used to wrap around a portion of a nerve that has not been transected-for example, a crushed or injured portion of nerve that otherwise remains intact.

While FIG. 2 depicts a nerve wrap device, comprising loops, needles, and hooks, other attachment features may be implemented as described further below. Further, while FIG. 2 depicts the incorporation of attachment features on two ends of nerve wrap device 206, various combinations of attachment features may be incorporated in a variety of positions on a nerve wrap device, some of which will be detailed further below.

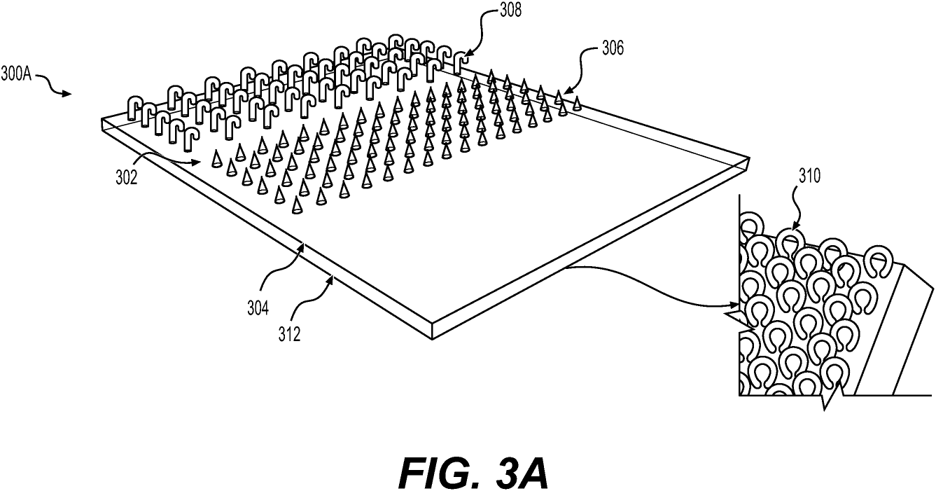

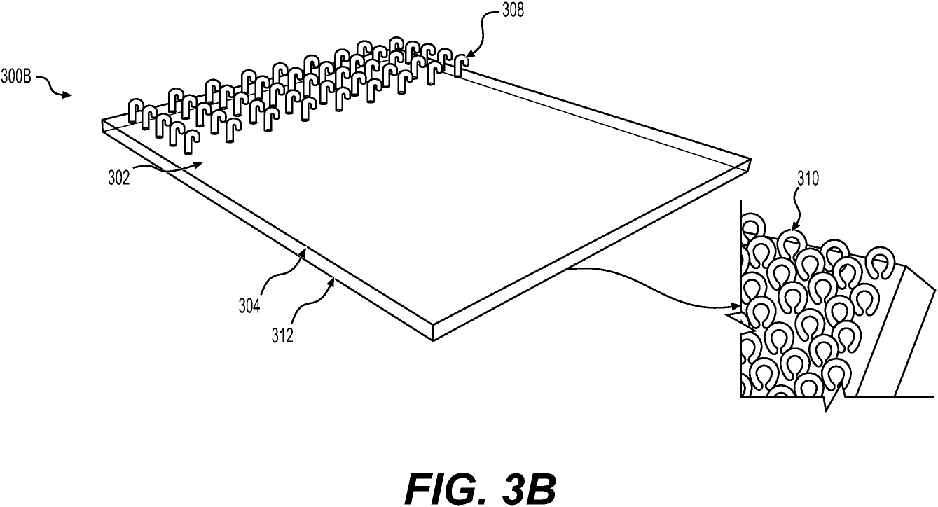

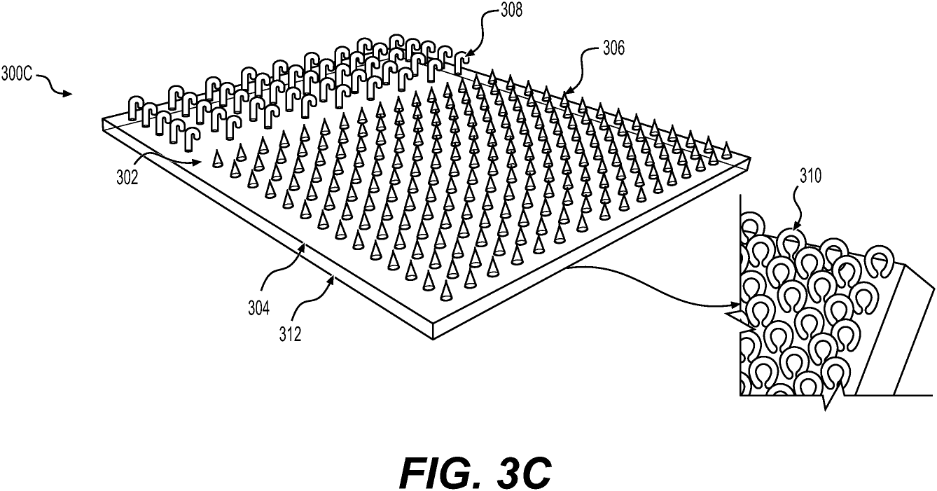

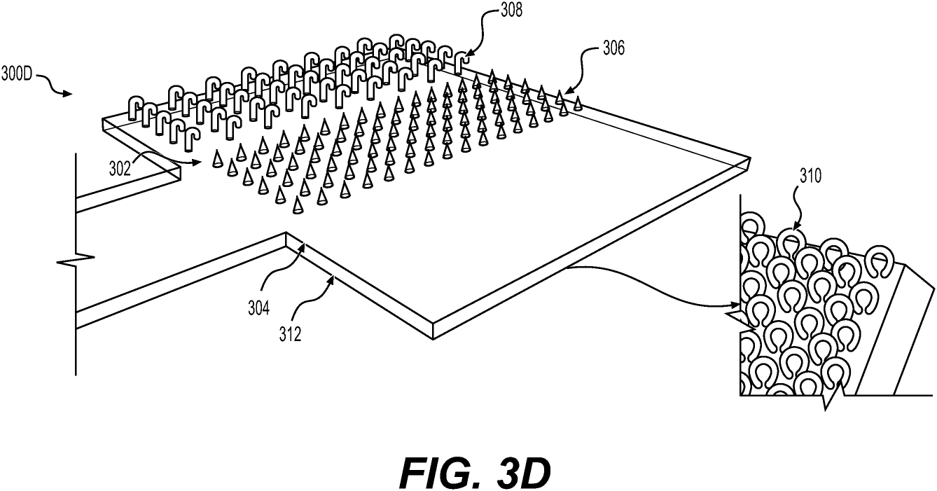

FIGS. 3A, 3B, 3C, and 3D illustrate variations of an exemplary nerve wrap device 300 (e.g., 300A, 300B, 300C, and 300D, respectively) including embedded hook and loop attachment features. Nerve wrap device 300A may be prepared from a membranous tissue graft in the form of sheet 302. Sheet 302 may include enough membranous tissue graft material to provide for outer portions that overlap with each other (i.e., overlapping portions) when the nerve wrap device 300A is wrapped around an injured nerve. The outer portions used for the overlapping configuration include a plurality of three dimensional (3D) attachment features embedded on sheet 302. The 3D attachment features on the outer portions of sheet 302 may be 3D printed attachment features or may be otherwise formed or secured to sheet 302. Furthermore, the attachment features embedded on the outer portions of sheet 302 may have complementary structures that allow the attachment features to interlock and adhere with one another when the respective outer portions overlap and come into contact.

A first, upper surface 304 includes a plurality of hooks 308 along an outer portion, and second, lower surface 312 includes a plurality of loops 310 along the opposite outer portion. FIGS. 3A-3D show a portion of lower surface 312 including loops 310, and it should be recognized that loops 310 may extend over a similar portion of lower surface 312 as compared to the portion over which hooks 308 extend over upper surface 304, loops 310 may extend over a comparatively narrower portion, or loops 310 may extend over a comparatively wider portion. Nerve wrap device 300A is configured so that the plurality of hooks 308 and the plurality of loops 310 are embedded on the outer portions of opposite surfaces, so that when the outer portions overlap the attachment features on one surface interface with the attachment features on the opposite, overlapping surface. Therefore, in some embodiments, the plurality of hooks 308 may be embedded on lower surface 312, and the plurality of loops may be embedded on upper surface 304. In order to facilitate engagement of hooks 308 with loops 310, the loops 310 may be configured to have more flexibility than hooks 308.

In some embodiments, sheet 302 may also include embedded 3D attachment features in the form of microneedles 306 ion at least a portion of the surface configured to contact the tissue when wrapped in place around the tissue. In FIG. 3A, upper surface 304 may be configured to contact the tissue during use. In the embodiment of FIG. 3A, microneedles 306 may be embedded on the center portion of sheet 302, so that they come in direct contact with the injured nerve. In particular, microneedles 306 may contact an outer region of the epineurium when the nerve wrap device 300A is positioned and wrapped around a nerve. Microneedles 306 may be considered tissue attachment features. The tapered ends of the microneedles 306 may allow the microneedles to attach to the epineurium to create friction to maintain nerve wrap device 300A in place relative to the nerve. Although FIG. 3A depicts microneedles 306 on the same surface of hooks 308 and on an opposite surface of loops 310, in some embodiments, microneedles 306 may be included on the same surface as loops 310 and on an opposite surface of hooks 308.

Nerve wrap device 300B as depicted in FIG. 3B may include similar hook and loop attachment features as nerve wrap device 300A. However, nerve wrap device 300B has been manufactured without microneedles. Accordingly, nerve wrap device 300B may function to adhere to itself to form a tubular structure when wrapping around a tissue but may not include microneedles to engage with the tissue around which the wrap is secured.

FIG. 3C depicts a nerve wrap device 300C that includes similar hook and loop attachment features and microneedles 306 as nerve wrap device 300A. In the embodiment of FIG. 3C, microneedles 306 extend from a central region of upper surface 304 to an edge of upper surface 304. In this embodiment, microneedles 306 may engage with a larger portion of tissue around which nerve wrap device 300C is wrapped compared to nerve wrap device 300A. In the embodiment of FIG. 3C, nerve wrap device 300C may be positioned around tissue so that microneedles 306 interface with a majority of a circumference of the tissue, or around the entire circumference of the tissue. Microneedles 306 may thus engage the tissue it surrounds, applying substantially even friction on the surfaces of the tissue that nerve wrap device 300C contacts. Most or an entire circumference of the tissue may contact sheet 302 where microneedles 306 are located. A portion of sheet 302 where hooks 308 are located may overlap with a portion of the opposite surface where loops 310 are located, so that hooks 308 engage and adhere to loops 310, as described above. As described above, although FIG. 3C depicts microneedles 306 on the same surface of hooks 308 and on an opposite surface of loops 310, in some embodiments, microneedles 306 may be included on the same surface as loops 310 and on an opposite surface of hooks 308.

FIG. 3D depicts a nerve wrap device 300D that includes similar hook and loop attachment features and microneedles 306 as nerve wrap device 300A. As seen in FIG. 2, attachment features, such as hooks 212, needles 210, and loops 208 may be incorporated at opposite ends of nerve wrap device 206. In use, ends of nerve wrap device 206 through which the nerve(s) will enter nerve wrap device 206 may include attachment features, while a middle portion of nerve wrap device 206 may not include attachment features and may be held in place by virtue of the attachment features located on the end regions. In other embodiments, such as those shown in FIGS. 3A through 3C, attachment features may be arranged over a majority of the length of upper and lower surfaces 304, 312, so that more of the portions configured to overlap with one another may include attachment features, and, if included, more of the portions of the wrap devices configured to interface with the tissue may include microneedles 306.

The device of FIG. 3D shows that, similar to FIG. 2, one or more end regions of nerve wrap device 300D may include attachment features, such as those described above. FIG. 3D depicts one end of nerve wrap device 300D including attachment features, which may also be included on an opposite end of nerve wrap device 300D (not shown). In some aspects, the ends of nerve wrap device 300D may be wider relative to a middle portion of nerve wrap device 300D that does not include attachment features in order to provide for overlapping regions of sheet 302 on which hooks 308 and loops 310 may be included. For example, a middle region of nerve wrap device 300D that does not include attachment features may not include portions configured to overlap with one another when nerve wrap device is positioned around tissue, whereas portions that incorporate attachment features may include portions configured to overlap. Accordingly, a middle portion without attachment features may be narrower relative to the end portions with attachment features. This is depicted in FIG. 3D, although differences in width may be exaggerated. In other aspects, however, as is shown schematically in FIG. 2, portions of nerve wrap devices without attachment features may not be different in width and may include portions that overlap when in a rolled configuration, even though attachment features are not included.

While FIG. 3D depicts a hook and loop and microneedle embodiment at end portions of nerve wrap device 300D, it is contemplated that any suitable embodiment described herein may be included at end regions. For example, arrangements such as those depicted in FIGS. 3B, 3C, 4, 5, or 6, or the modifications described herein, may be included at end regions but not at middle regions.

Further, although FIGS. 3A-3D depict a plurality of hooks 308 and a plurality of loops 310 spanning across several rows, in at least one embodiment, the size of the respective attachment features may be increased and fewer attachment features having a larger size may be embedded on the outer portions of the opposite surfaces of nerve wrap device 300. In some examples, nerve wrap device 300 may be configured to have only one hook 308 and one loop 310 embedded on the outer portions of the opposite surfaces. In these examples, the one hook 308 and the one loop 310 may be configured to have a size that is large enough to provide an attachment strength that is suitable for connecting the overlapping surfaces and holding the nerve wrap device together.

Although not shown in the figures, it is also contemplated that some attachment features, for example tissue attachment features, such as microneedles, may be included only at one or more end portions, but attachment features configured to engage one another (e.g., hook and loop attachment features or others described herein) may extend along a majority of, or the entirety of, a length of opposite portions of a nerve wrap that are configured to overlap with one another. For example, in regards to FIG. 2, this embodiment may be altered so that regions of hooks 212A and 212B may extend towards one another, creating a continuous outer region of hooks. On the opposite side, regions of loops 208A and 208B may also extend towards one another, creating a continuous outer region of loops, whereas needles 210A and 210B may only be located on the end portions, as shown in FIG. 2.

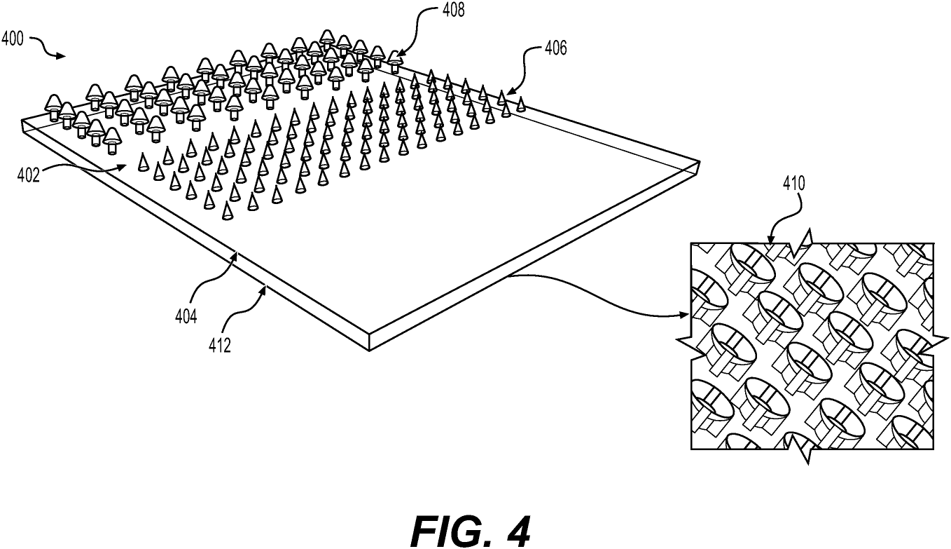

FIG. 4 illustrates an exemplary nerve wrap device 400 including embedded ball and socket attachment features, as well as microneedle attachment features. Nerve wrap device 400 may be prepared from a membranous tissue graft in the form of sheet 402. Sheet 402 may include outer portions that are configured to overlap with each other (i.e., overlapping portions) when the nerve wrap device 400 is wrapped around an injured nerve. Each outer portion configured to overlap includes a plurality of three dimensional (3D) attachments embedded on sheet 402. The 3D attachment features on the outer portions of sheet 402 may be 3D printed attachment features or may be otherwise formed or secured to sheet 402. Furthermore, the attachment features embedded on the outer portions of sheet 402 may have complementary structures that allow the attachment features to interlock and adhere with one another when the respective outer portions overlap and come into contact.

A first, upper surface 404 includes a plurality of ball portions 408 along an outer portion, and second, lower surface 412 includes a plurality of sockets 410 along the opposite outer portion. It is recognized that although the term ball and socket is used to refer to the complimentary attachment features in FIG. 4D, ball portions 408 and sockets 410 may have any suitable complimentary shapes that allow features 408 to releasably engage features 410. For example, ball portions 408 may be conical, spherical, ellipsoid, convex, pulvinate, ovoid, parabolic, cylindric, cuspidate, campanulate, umbonate, papillate, or funnel-shaped. Sockets 410 may be configured (e.g., dimensioned and shaped) to releasably receive and retain the ball portion upon contact. In some embodiments, sockets 410 may extend completely through sheet 402, while in other embodiments, sockets 410 may extend only partially through sheet 402.

For example, nerve wrap device 400 is configured so that the plurality of ball portions 408 and the plurality of sockets 410 are embedded on the outer portions of opposite surfaces, so that when the outer portions are overlapped, the attachment features on one surface interface with and adhere to the attachment features on the opposite, overlapping surface. Therefore, in some embodiments the plurality of ball portions 408 may be embedded on lower surface 412, and the plurality of sockets 410 may be embedded on upper surface 404. Although FIG. 4 depicts a plurality of ball portions 408 and a plurality of sockets 410 spanning across several rows, in at least one embodiment, the size of the respective attachment features may be increased and fewer attachment features having a larger size may be embedded on the outer portions of the opposite surfaces of nerve wrap device 400. In some examples, nerve wrap device 400 may be configured to have only one ball portion 408 and one socket 410 embedded on the outer portions of the opposite surfaces. In these examples, the one ball portion 408 and the one socket 410 may be configured to have a size that is large enough to provide an attachment strength that is suitable for connecting the overlapping surfaces and holding the nerve wrap device together.

Sheet 402 may also include embedded 3D attachment features in the form of microneedles 406 in the center portion. Microneedles 406 may function in a similar manner (e.g., attaching to an outer portion of the tissue around which device 400 is wrapped, such as the epineurium in the case of nerves) as the microneedles 306 described above in reference to FIG. 3A. As described above, although FIG. 4 depicts microneedles 406 on the same surface of ball portions 408 and on an opposite surface of sockets 410, in some embodiments, microneedles 406 may be included on the same surface as sockets 410 and on an opposite surface of ball portions 408. Further, although not shown, it is contemplated that ball and socket attachment features may be arranged as is shown in FIG. 4, or may replace the hook and needle attachment features and may be arranged as is shown in FIGS. 3B through 3D, including without microneedles as in FIG. 3B, or as described in reference to the hook and loop embodiments described above.

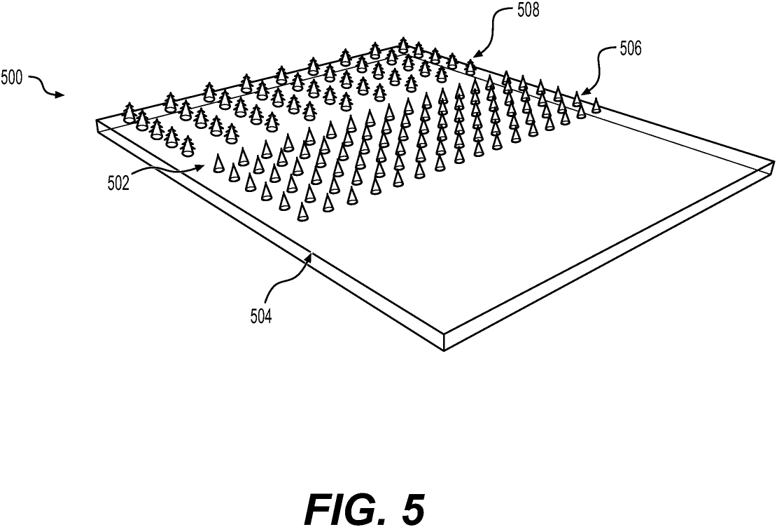

FIG. 5 illustrates an exemplary nerve wrap device 500 including embedded barbed microneedles and microneedle attachment features. Nerve wrap device 500 may be prepared from a membranous tissue graft in the form of sheet 502. Sheet 502 may include outer portions that are configured to overlap with each other (i.e., overlapping portions) when the nerve wrap device 500 is wrapped around an injured nerve. Unlike previously described devices, sheet 502 of nerve wrap device 500 may only include 3D attachment features on one outer portion used for overlapping, as opposed to both outer portions. For example, a plurality of barbed microneedles 508 are embedded on one outer portion of sheet 502, with no complimentary attachment features included on a portion of the opposite surface that abuts barbed microneedles 508 when overlapped with one another. Barbed microneedles 508 serve as a variation of the microneedles 306 and 406 previously described in reference to FIGS. 3A and 4. Barbed microneedles 508 each include a plurality of barbs protruding from the surface of each microneedle. The barbs may be angled so that a free end of each barb is angled towards sheet 502. When the outer portions of sheet 502 are wrapped around an injured nerve and overlap, the barbs on the barbed microneedles 508 may be embedded into one of the outer portions on the opposite side of sheet 502. Once embedded into the opposite surface, the barbs on barbed microneedles 508 may catch and latch onto the opposite outer portion when they overlap.

Sheet 502 may also include microneedles 506, without barbs, as described above in reference to the embodiments of FIGS. 2, 3A, 3C, 3D, and 4. Microneedles 506 may function in a similar manner (e.g., attaching to an outer portion of the tissue around which device 400 is wrapped, such as the epineurium in the case of nerves) as the microneedles 306 described above in reference to FIG. 3A. In other embodiments, however, barbed microneedles 508 may be used as an alternative to microneedles 306, 406 or may be interspersed with microneedles 306, 406, 506. For example, instead of incorporating microneedles 506 as is shown in FIG. 5, barbed microneedles 508 may instead be incorporated where microneedles 506 are depicted, so that microneedles 508 extend across a majority of one side of sheet 502.

Further, although not shown, it is contemplated that the barbed microneedle attachment features may be arranged as is shown in FIG. 5, or may replace the hook and needle attachment features and may be arranged as is shown in FIGS. 3B through 3D, including without microneedles as in FIG. 3B, or as described in reference to the hook and loop embodiments described above. The only difference is that barbed microneedles 508 may not require a complimentary attachment feature on an opposite surface configured for overlapping with the portion on which barbed microneedles 508 are incorporated. Further, in each of the various embodiments described herein, barbed microneedles 508 may be used in place of, or in addition to, un-barbed microneedles.

Although FIG. 5 depicts a plurality of barbed microneedles 508 spanning across several rows, in at least one embodiment, the size of the barbed microneedles 508 may be increased and fewer barbed microneedles 508 having a larger size may be embedded on the outer portion of nerve wrap device 500. In some examples, nerve wrap device 500 may be configured to have only one barbed microneedle 508. In these examples, the one barbed microneedle 508 may be configured to have a size that is large enough to provide an attachment strength that is suitable for connecting the overlapping surfaces and holding the nerve wrap device together.

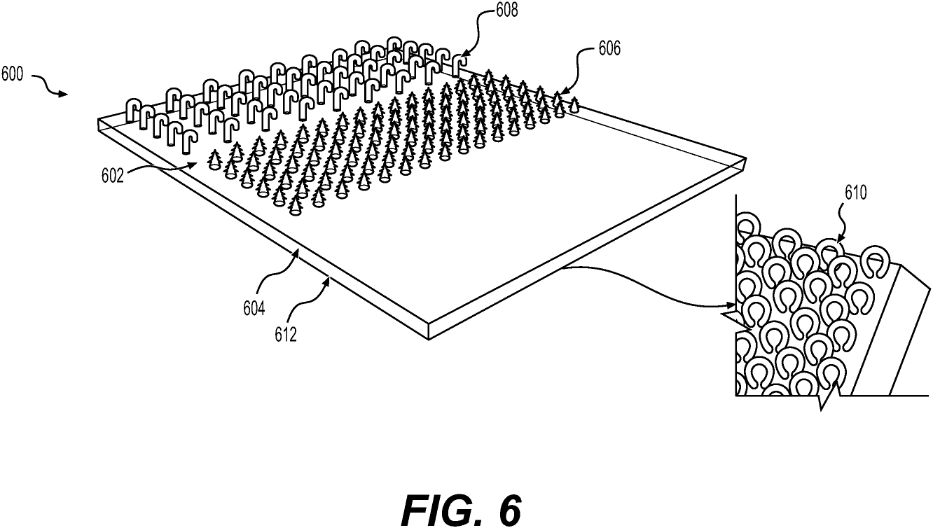

FIG. 6 depicts another exemplary nerve wrap device 600 containing embedded hook and loop attachment features with barbed microneedles 606 incorporated in place of un-barbed microneedles, as alluded to immediately above. The hook and loop attachment features of nerve wrap device 600 may have a similar configuration to the hook and loop attachment features of nerve wrap devices 300A, 300B, 300C, and 300d, as described above. However, sheet 602 of nerve wrap device 600 includes embedded 3D attachment features in the form of barbed microneedles 606 in a central portion. Barbed microneedles 606 each include a plurality of barbs protruding from the surface of each microneedle. The barbs may be angled so that a free end of each barb is angled towards sheet 602. Barbed microneedles 606 may function in a similar manner as microneedles 306, but in some aspects, may create a more secure attachment to the tissue nerve wrap device 600 is configured to wrap around, due to the catching of the barbs within the tissue, which may inhibit the wrap from pulling away from the tissue.

For example, barbed microneedles 606 may contact the epineurium when the nerve wrap device 600 is wrapped around a nerve. The tapered points of the barbed microneedles 606 may allow the barbed microneedles to pierce into to the epineurium like the microneedles without barbs. Moreover, in some aspects, the barbs of barbed microneedles 606 may be capable of flexing when the barbed microneedles 606 penetrate the nerve tissue. The flexing may allow the barbed microneedles 606 to pierce into the tissue, and, once in the tissue, the barbs may then flex outwards when the tissue and the wrap are pulled away from each other, inhibiting removal of the wrap from the tissue. In other words, barbed microneedles according to aspects of the present disclosure may provide sufficient attachment and anchoring of the nerve wrap device at the epineurium without penetrating too deep and causing further nerve damage.

In some embodiments of the present disclosure, microneedles and/or barbed microneedles as described in reference to FIGS. 2, 3A, and 4-6 above, may be configured to provide additional attachment properties for adhering the nerve wrap device to itself and/or for adhering to the nerve. The microneedles and/or barbed microneedles may have certain features (e.g., a hollow structure) that allow substances to be stored within. In certain examples, the microneedles and/or barbed microneedles may be configured to store and release chemical adhesives. For example, the microneedles and/or barbed microneedles may be loaded with a chemical adhesive and configured to release said adhesive when pressure is applied upon contact with the tissue, the overlapping portion of the nerve wrap, or both. Alternatively, the microneedles and/or barbed microneedles may be configured to have a tip or covering that is configured to degrade and allows for the release of the chemical adhesive stored within the microneedles based on the passage of time or exposure to a reactant. In still other aspects, the microneedles and/or barbed microneedles may be configured to have a tip or covering that is configured to mechanically separate from the microneedles when pushed into the tissue. Such embodiments may enhance the adhesive properties by providing both mechanical and chemical adhesion.

Further, the configurations for microneedles and/or barbed microneedles that allow for the release of substances may also provide additional therapeutic properties to the injured nerve. For example, the pressure release, degradation, or mechanical separation mechanisms as described above with respect to the release of chemical adhesives may also provide for the release of drug formulations that may be used to treat the injured nerve upon release.

Each of the 3D attachment features described above and shown in FIGS. 3A-3D and 4-6 may be sized on the order of micrometers to correspond with the dimensions of the membranous tissue graft used for the nerve wrap device. As discussed above, certain 3D attachment features may be sized to be small enough to allow for a plurality of attachment features (e.g., in multiple rows) to be embedded on the outer portions of the opposite surfaces of the nerve wrap device or large enough to allow for less attachment features to be embedded on the outer portions of the opposite surfaces of the nerve wrap device. An exemplary diameter range for the 3D attachment features according to the embodiments of the present disclosure may be about 1000 µm (1 mm) or less, for example, from about 0.1 µm to about 1000 µm, from about 1 µm to about 500 µm, from about 5 µm to about 250 µm, from about 10 µm to about 150 µm, or from about 25 µm to about 100 µm. An exemplary height range for the 3D attachment features according to the embodiments of the present disclosure may be about 1000 µm (1 mm) or less, for example, from about 1 µm to about 250 µm, from about 3 µm to about 200 µm, from about 5 µm to about 150 µm, from about 8 µm to about 100 µm, or from about 10 µm to about 50 µm. In particular, microneedles and/or barbed microneedles of the nerve wrap device configured for contact and attachment with the epineurium may be sized in a range that allows the microneedles and/or barbed microneedles to penetrate no more than a depth of about 200 microns, for example, about 150 microns or less, although this may depend at least in part on the tissue that the wrap is configured for use with. An exemplary diameter range for the microneedles and/or barbed microneedles of the present disclosure may be from about 25 µm to about 75 µm. In some examples, the diameter for the microneedles and/or barbed microneedles may be about 50 µm.

In addition, the attachment features described above for one embodiment may be combined with any other attachment features of a different embodiment. Thus, any combination of attachment features may be used. For example, the ball and socket configuration may be applied to the outer portions of a nerve wrap for overlapping and the central portion may not include microneedles. Further, various combinations of attachment features may be used for the same nerve wrap device. In some embodiments, combinations of hooks and balls may be included on one surface of an outer portion of the nerve wrap, while combinations of loops and sockets are included on the opposite surface of the other outer portion. Microneedles and barbed microneedles may be embedded on the center portion of the nerve wrap in an alternating configuration or may be interspersed with one another. In some embodiments, a plurality of the same 3D attachment features and/or different 3D attachment features may have a uniform size, while in other embodiments the sizes of the same 3D attachment features and/or different 3D attachment features may vary. Further, although the attachment features are depicted in the figures as being arranged in rows and columns, the attachment features may be regularly or irregularly spaced in any suitable arrangement. In some aspects, there may be one or more clusters of attachment features included on outer portions, or there may be fewer attachment features, e.g., from about 1 to about 10 attachment features.

The attachment features, as described above, may be formed via additive manufacturing. In some embodiments, the attachment features may be manufactured via 3D printing. The 3D printing may refer to sequential addition of biocompatible material layers or joining of biocompatible material layers (or parts of biocompatible material layers) to form a 3D structure. Suitable 3D printing devices that may be used to manufacture the attachment features according to embodiments of the present disclosure may include, but are not limited to, stereolithographic printers and multiphoton lithography printers.

The biocompatible material (or bioink) used for the 3D printing of the attachment features may include natural or synthetic structural proteins, such as fibrinogen, albumin, fibronectin, collagen, decellularized ECMs, or hyaluronic acid; hydrogels; biodegradable polymers and copolymers; living biological components, such as undifferentiated stem cells, partially differentiated stem cells, terminally differentiated cells, microvascular fragments, or organelles; and/or macromolecules. Exemplary polymers and copolymers may include, but are not limited to, polyurethane, polyurethane/urea, poly(glycolic acid), poly(lactic acid), poly(lactic-co-glycolic acid), polycaprolactone, poly(ethylene glycol) diacrylate (PEGDA), and mixtures thereof. In some aspects, post-processing may occur. For example, the printed attachment features may be further crosslinked either during the printing process or following printing, e.g., via drying, heating, or ultraviolet or visible irradiation.

In some examples, the attachment features of the present disclosure may be prepared through 3D printing of poly(ethylene glycol) diacrylate (PEGDA). Further, a photoinitiator, such as Lithium phenyl-2, 4, 6-trimethylbenzoylphosphinate (LAP), may be used to crosslink PEDGA during or following the 3D printing process.

Materials used to form the attachment features described herein may be formed of a degradable biocompatible material. Accordingly, after a given amount of time, nerve wrap devices of the present disclosure may degrade. In some aspects, nerve wrap devices may be configured to degrade after, e.g., a period of about 4 weeks to about 24 weeks, e.g., from about 8 weeks to about 20 weeks, from about 10 weeks to about 20 weeks, or about 14 weeks to about 18 weeks. In some aspects, nerve wrap devices may degrade after about 12 weeks, after about 14 weeks, after about 16 weeks, after about 18 weeks, or after about 20 weeks. In addition to the entire nerve wrap device being configured to degrade after a period of about 4 weeks to about 24 weeks as discussed above, in certain embodiments, certain attachment features may also have a specific period of degradation. For example, microneedles and/or barbed microneedles that are loaded with a chemical adhesive and that have a component (e.g., a tip or covering) that is configured to degrade to allow for the release of said adhesive, may be configured to degrade after a period of less than a few days, e.g., a period of about 1 hour to about 48 hours. Embodiments including microneedles and/or barbed microneedles that are not loaded with a chemical adhesive, may also have a relatively shorter degradation time compared to the rest of the nerve wrap device, e.g., on the order of hours or days. For example, the microneedles and/or barbed microneedles may be configured to degrade after a period of less than a few days, e.g., a period of about 1 hour to about 48 hours.

In some aspects of the present disclosure, the attachment features (e.g., hooks and loops, microneedles, and balls and sockets) may be 3D printed directly onto the membranous tissue graft material (e.g., small intestine submucosa (SIS) material) used for the nerve wrap. Other embodiments of the present disclosure may include combinations of 3D printing and vacuum pressing and/or molding, e.g., micro molding, the attachment features directly onto the sheet. In some examples, the desired attachment features may be embedded into layers of the membranous tissue graft material (e.g., SIS material), and the layers may subsequently be vacuum pressed together. For example, attachment features may be printed or otherwise attached onto a sheet of tissue graft material, and then additional layers of the sheet of tissue graft material may be vacuum pressed over the printed attachment features so that the attachment features protrude through or otherwise project out from the one or more layers vacuum sealed over the attachment features. In certain examples, an adhesive may be used to add the separately prepared 3D printed attachment features on the surface of the membranous tissue graft in sheet form. Another technique for adhering certain attachment features (e.g., hooks, loops, balls, barbed or un-barbed microneedles) to the surface of the membranous tissue graft may include using barbs to hold the 3D attachment features in place on the membranous tissue graft.

In other aspect, portions of the attachment features may be made by subtractive manufacturing. For example, sockets may be formed by removing material from a membranous tissue graft. In some examples, portions of the attachment features may be made by multiphoton lithography. For example, multiphoton lithography may be used to uncrosslink (or unlink) certain areas at the surface of a membranous tissue graft, which may be removed (e.g., washed away) from the membranous tissue graft to leave behind a desired shape (e.g., sockets). In yet another aspect, portions of the attachment features may be formed by a combination of molding and cutting out the molded attachment features. In some examples, sockets may be formed by creating a mold with desired spikes and pressing a membranous tissue graft or a biocompatible material in sheet form onto the mold containing the spikes to cut out the sockets.

Some aspects of the present disclosure may provide methods of repairing injured nerves, particularly, damaged or severed nerves. In these methods, nerve wrap devices in the form of a sheet comprising the 3D attachment features as described above, may be wrapped around the injured nerve. Use of such a nerve wrap device may allow the body’s natural healing process to repair the severed nerve by isolating and protecting the severed nerve during the healing process. For example, the patient’s cells can incorporate into the extracellular matrix to remodel and form a tissue similar to the nerve epineurium.

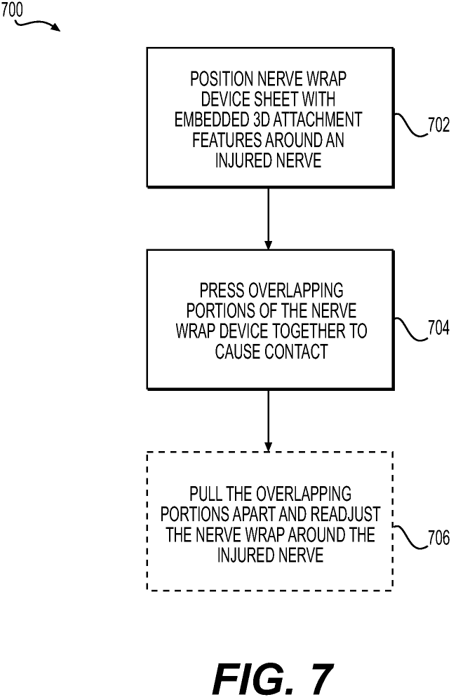

While aspects of the use of exemplary nerve wrap devices have already been described herein, FIG. 7 depicts, in flow chart form, an exemplary general method 700 for using a nerve wrap device according to aspects of the present disclosure. Method 700, and variations thereof, may be applicable to any nerve wrap device described or encompassed by this disclosure. It will be contemplated by those of ordinary skill in the art that FIG. 7 depicts merely an exemplary method, of which many variations are possible. In some embodiments, one or more steps of FIG. 7 may be added, removed, duplicated, or performed out of order. The steps of method 700, and variations thereon, may be performed by a physician, such as an access surgeon or other surgeon performing microsurgery (e.g., nerve reconstruction surgery, peripheral nerve repair surgery, etc.). Further, although FIG. 7 is in reference to a nerve wrap device for attaching around a nerve, FIG. 7 may be a generic tissue wrap device for repair or reconstruction of any suitable tissue.

According to step 702 of method 700, a nerve wrap device in a sheet configuration with embedded 3D attachment features may be positioned around an injured nerve. This step may be performed by, e.g., a surgeon performing microsurgery. In step 704, overlapping portions of the nerve wrap device may be pressed together to cause contact. In optional step 706, the overlapping portions may be pulled apart and readjusted around the injured nerve. If optional step 706 is performed, then step 704 may be performed again once the overlapping portions are readjusted. In some examples, a tool, such as forceps or other suitable clamping mechanism, may be used to press the overlapping portion of the nerve wrap device together and/or pull them apart. If a tool is used, a force may be applied only to the overlapping portions of the nerve wrap device, and not the nerve or other tissue around which the device is wrapped. For example, a portion of the tool may be positioned between the nerve and overlapping portions of the device, another portion of the tool may be positioned on an opposite side of the overlapping portions. In other examples, after steps 704 and/or 706 are performed, one or more sutures may be used to, e.g., attach the nerve wrap device to the nerve and/or to reinforce attachment of the attachment features.

While principles of the present disclosure are described herein with reference to illustrative aspects for particular applications, it should be understood that the disclosure is not limited thereto. Those having ordinary skill in the art and access to the teachings provided herein will recognize additional modifications, applications, aspects, and substitution of equivalents all fall within the scope of the aspects described herein. Furthermore, the present disclosure is not limited to the exemplary shapes, sizes, and/or materials discussed herein. Thus, a person of ordinary skill in the art will recognize that additional shapes, sizes, and/or materials may be used as discussed herein to achieve the same or similar effects or benefits as discussed above. Accordingly, the present disclosure is not to be considered as limited by the foregoing description.

What is claimed is:

1. A tissue wrap device, the tissue wrap device comprising:
a sheet of biocompatible material, the sheet having a first side, a second side, a middle portion, a first outer portion, and a second outer portion, the first side of the first outer portion being configured to overlap and interface with the second side of the second outer portion when the sheet is transitioned to a rolled configuration;
wherein the first side of the first outer portion comprises a plurality of three dimensional attachment features; and
wherein the plurality of three dimensional attachment features are configured to engage with the second side of the second outer portion to maintain the sheet in the rolled configuration.

2. The tissue wrap device of claim 1, wherein the plurality of three dimensional attachment features is a first plurality of three dimensional attachment features, and wherein the second side of the second outer portion comprises a second plurality of three dimensional attachment features.

3. The tissue wrap device of claim 1, further comprising a plurality of barbed or un-barbed microneedles located on the middle portion.

4. The tissue wrap device of claim 3, wherein the barbed or un-barbed microneedles are hollow and are configured to release one or more of a chemical adhesive or a drug formulation.

5. The tissue wrap device of claim 3, wherein the barbed or un-barbed microneedles have a height of 150 µm or less.

6. The tissue wrap device of claim 1, wherein the sheet is made from a material selected from one or more of: small intestine submucosa, amniotic/chorionic membrane, reconstituted denatured collagen, collagen, elastin, thrombin, fibronectin, starches, poly(amino acid), gelatin, alginate, pectin, fibrin, oxidized cellulose, chitin, chitosan, tropoelastin, hyaluronic acid, fibrin-based materials, collagen-based materials, hyaluronic acid-based materials, glycoprotein-based materials, cellulose-based materials, and silk.

7. The tissue wrap device of claim 1, wherein the plurality of three dimensional attachment features are 3D printed.

8. The tissue wrap device of claim 1, wherein the three dimensional attachment features are made from one or more of polyurethane, polyurethane/urea, poly(glycolic acid), poly(lactic acid), poly(lactic-co-glycolic acid), polycaprolactone, and poly(ethylene glycol) diacrylate (PEGDA).

9. The tissue wrap device of claim 1, wherein at least some of the three dimensional attachment features have a diameter of about 25 µm to about 75 µm.

10. The tissue wrap device of claim 1, wherein at least some of the three dimensional attachment features have a height in a range of about 1000 µm or less.

11. The tissue wrap device of claim 1, wherein the plurality of three dimensional attachment features on the first side of the first portion include barbed microneedles.

12. The tissue wrap device of claim 11, wherein the barbed microneedles are hollow and are configured to release one or more of a chemical adhesive or a drug formulation.

13. The tissue wrap device of claim 11, wherein the second side of the second portion does not include any three dimensional attachment features.

14. The tissue wrap device of claim 11, wherein the middle portion comprises a plurality of barbed or un-barbed microneedles.

15. The tissue wrap device of claim 1, wherein the plurality of three dimensional attachment features is a first plurality of three dimensional attachment features, wherein the second side of the second outer portion comprises a second plurality of three dimensional attachment features, and wherein the first plurality of three dimensional attachment features includes ball portions, and the second plurality of three dimensional attachment features includes socket portions.

16. The tissue wrap device of claim 15, wherein the middle portion comprises a plurality of barbed or un-barbed microneedles.

17. The tissue wrap device of claim 1, wherein the plurality of three dimensional attachment features is a first plurality of three dimensional attachment features, wherein the second side of the second outer portion comprises a second plurality of three dimensional attachment features, and wherein the first plurality of three dimensional attachment features includes hooks, and the second plurality of three dimensional attachment features includes loops.

18. The tissue wrap device of claim 17, wherein the middle portion comprises a plurality of barbed or un-barbed microneedles.

19. A method of repairing a tissue, comprising wrapping the tissue wrap device of claim 1 around the tissue, and overlapping the first side of the first outer portion with the second side of the second outer portion, wherein the plurality of three dimensional attachment features adheres the first side of the first outer portion to the second side of the second outer portion to maintain the tissue wrap device in the rolled configuration.

20. A method of manufacturing a plurality of three-dimensional attachment features for a tissue wrap device, the method comprising:
three dimensional (3D) printing a biocompatible material to form a plurality of three dimensional attachment features, the biocompatible material comprising at least one of polyethylene glycol) diacrylate, polyurethane, polyurethane/urea, poly(glycolic acid), poly(lactic acid), poly(lactic-co-glycolic acid), polycaprolactone, agarose, alginate, chitosan, collagen, fibrin, gelatin, hyaluronic acid, gelatin methacryloyl, polyethylene glycol, or a mixture thereof;
wherein the three dimensional attachment features have an average diameter of about 25 µm to about 75 µm and a height of about 1000 µm or less.

21. The method of claim 20, further comprising adhering the three dimensional attachment features to a sheet of membranous tissue graft to form the tissue wrap device.

22. The method of claim 21, wherein adhering comprises using a chemical adhesive to adhere the three-dimensional attachment features to the sheet of membranous tissue graft.

23. The method of claim 21, wherein adhering comprises vacuum pressing the three dimensional attachment features to the sheet of membranous tissue graft.

24. The method of claim 20, wherein the three dimensional attachment features are 3D printed onto a sheet of membranous tissue graft.

25. A method of using a nerve wrap device to protect a damaged nerve, the method comprising:
positioning the nerve wrap device relative to the damaged nerve; and
wrapping the nerve wrap device around the nerve so that a first portion of the nerve wrap device overlaps a second portion of the nerve wrap device, forming a rolled configuration of the nerve wrap device, wherein at least one of the first portion and the second portion includes a plurality of three dimensional attachment features, and wherein the plurality of three dimensional attachment features secures the first portion and the second portion to each other to maintain the rolled configuration.

26. The method of claim 25, wherein wrapping the nerve wrap device around the nerve so that the first portion overlaps the second portion includes pressing the first portion and the second portion together.

27. The method of claim 26, further comprising using a tool to press the first portion and the second portion together.

28. The method of claim 25, wherein the method does not comprise suturing the nerve wrap device in place.
